(19) United States
(12) Patent Application Publication
(10) Pub. No.: US 2026/0209841 A1
(43) Pub. Date: Jul. 23, 2026

(54) SYSTEMS AND METHODS FOR MINIMAL RESIDUAL DISEASE ANALYSIS

(71) Applicant: Predicine, Inc., Hayward, CA (US)

(72) Inventors: Pan DU, Dublin, CA (US); Shidong JIA, Palo Alto, CA (US)

(21) Appl. No.: 19/297,727

(22) Filed: Aug. 12, 2025

Related U.S. Application Data
(63) Continuation of application No. PCT/US2024/015383, filed on Feb. 12, 2024.
(60) Provisional application No. 63/445,151, filed on Feb. 13, 2023.

(51) Int. Cl. C12Q 1/6869; C12Q 1/6886; G16B 20/20; G16B 25/10; G16H 10/40; G16H 10/60; G16H 50/20

(52) U.S. Cl. CPC C12Q 1/6869; C12Q 1/6886; G16B 20/20; G16B 25/10; G16H 10/40; G16H 10/60; G16H 50/20; C12Q 2600/112; C12Q 2600/156; C12Q 2600/158

(57) ABSTRACT

Provided herein are methods and systems for detection and monitoring of cancer. The method may comprise minimal residual disease analysis. The method may comprise using nucleic acids from a urine sample or urine cell pellet sample. The methods may comprise assaying nucleic acids in urine to detect a set of biomarkers from samples. The method may comprise monitoring a subject after a transurethral resection of bladder tumor.

### CROSS-REFERENCE

This application is a continuation of International Application No. PCT/US2024/015383, filed Feb. 12, 2024, which claims the benefit of U.S. Provisional Application No. 63/445,151, filed Feb. 13, 2023, each of which is incorporated by reference herein in its entirety.

### BACKGROUND

Cancer is a leading cause of deaths worldwide. Detection of cancer in individuals may be critical for providing treatment and improving patient outcomes. Cancer may be caused by genetic aberration which may lead to unregulated growth of calls. Detection of the genetic aberrations may be important for the detection of cancer. Sequencing of nucleic acids in a sample from a patient may be used to detect genetic aberrations.

### SUMMARY

Provided herein are systems and methods for detection of the presence or absence of cancer in a subject. The systems and methods provided herein comprises assaying polynucleotides to identify biomarkers of cancers in a subject. Detection of a type of cancer or the specific biomarkers for a given cancer may allow an effective treatment to be provided to an individual and may result in improved outcomes. For multiple types of cancer, the particular biomarkers that indicate a particular cancer type (or subtype) may be used to identify a prognosis for an individual suffering from the cancer. In order to provide accurate detection and prognosis for a cancer, multiple analytes may be examined. By analyzing an increased number of analytes (and sets of biomarkers from the analytes), the detection of a cancer (or cancer parameter) may be improved and may allow for the recommendation of an effective treatment, and may also allow for the prognosis to be more accurate.

In an aspect, the present disclosure provides a method for identifying presence or an absence of minimal residual disease (MRD) in a subject, comprising: (a) assaying deoxyribonucleic acid (DNA) molecules from a first biological sample obtained or derived from the subject at a first time point (b) detecting a set of biomarkers from the DNA molecules based at least in part on the assaying of (a), wherein the set of biomarkers comprise differentially expressed markers or variants; (c) generating a plurality of probe nucleic acids that are customized for the subject, wherein the probe nucleic acids comprises sequences of at least a subset of the set of biomarkers; (d) using the plurality of probe nucleic acids, sequencing cell DNA from a second biological sample obtained or derived from the subject at a second time point to detect the presence or absence of the subset of the set of biomarkers, wherein the sequencing is performed at a depth of at least 80×, wherein the second biological sample is a blood sample, a urine sample, or a urine cell pellet sample; (e) computer processing the subset of the set of biomarkers to detect the presence or absence of minimal residual disease (MRD) in the subject. In some embodiments, the first biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof. In some embodiments, the first biological sample comprises the plasma sample. In some embodiments, the first biological sample comprises the urine sample. In some embodiments, the first biological sample comprises the tumor tissue sample. In some embodiments, the first or second biological sample is obtained or derived from the subject using an ethylenediaminetetraacetic acid (EDTA) collection tube, a cell-free RNA collection tube, or a cell-free deoxyribonucleic acid (DNA) collection tube, other blood collection tube, and CTC collection tubes. In some embodiments, (a) comprises subjecting the first or second biological sample to conditions that are sufficient to isolate, enrich, or extract the DNA molecules In some embodiments, the method further comprises fractionating the first biological sample of the subject to obtain the DNA molecules, wherein the first biological sample is a whole blood sample. In some embodiments, at least one of the DNA molecules are assayed using DNA sequencing to produce nucleic acid sequencing reads. In some embodiments, the DNA sequencing comprises whole exome sequencing. In some embodiments, the method further comprises filtering at least a subset of the nucleic acid sequencing reads based on a quality score. In some embodiments, the method further comprises performing error correction on the nucleic acid sequencing reads using sample barcodes or molecular barcodes attached to at least one of the DNA molecules. In some embodiments, the method further comprises performing at least one of single-stranded consensus calling and double-stranded consensus calling on the nucleic acid sequencing reads, thereby suppressing sequencing and PCR errors in the nucleic acid sequencing reads. In some embodiments, the sequencing of (d) is performed at a depth of at least 100×. In some embodiments, the sequencing of (d) is performed at a depth of at least 1,000×. In some embodiments, the sequencing of (d) is performed at a depth of at least 10,000×. In some embodiments, the sequencing of (d) is performed at a depth of at least 100,000×. In some embodiments, the sequencing of (e) comprises sequencing nucleic acids derived from the first biological sample. In some embodiments, the sequencing of (e) comprises sequencing nucleic acids derived from the second biological sample. In some embodiments, the sequencing of (e) comprises sequencing nucleic acids of a sample taken at the first time point, and sequencing nucleic acids of a sample taken at a second time point. In some embodiments, the assaying of (a), sequencing of (d), or sequencing of (e) comprises nucleic acid amplification. In some embodiments, the nucleic acid amplification comprises polymerase chain reaction (PCR) or isothermal amplification. In some embodiments, the cancer is selected from the group consisting of: genitourinary cancer, prostate cancer, bladder cancer, and any combination thereof. In some embodiments, the cancer comprises the bladder cancer. In some embodiments, the bladder cancer is a muscle invasive bladder cancer. In some embodiments, the subject is asymptomatic for the cancer.

In some embodiments, the method comprises detecting the presence or absence of minimal residual disease in the subject at an accuracy of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a sensitivity of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a specificity of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject at a positive predictive value of at least about 60%, at least about 70%, at least about 75%, at least about 80%, at least about 85%, at least about 90%, at least about 95%, at least about 98%, or at least about 99%. In some embodiments, the method comprises detecting the presence or absence of minimal residual disease in the subject in the subject at a negative predictive value of at least about 60%, at least about 70%, at least about 75%, at least about 80%, at least about 85%, at least about 90%, at least about 95%, at least about 98%, or at least about 99%. In some embodiments, the first biological sample is obtained or derived from the subject prior to the subject receiving a therapy for the cancer. In some embodiments, the biological sample is obtained or derived from the subject during a therapy for the cancer. In some embodiments, the biological sample is obtained or derived from the subject after receiving a therapy for the cancer. In some embodiments, the therapy is selected from the group consisting of: surgical resection, chemotherapy, radiotherapy, immunotherapy, cell therapy, adjuvant therapy, neoadjuvant therapy, androgen deprivation therapy, and a combination thereof. In some embodiments, the first biological sample is obtained or derived from the subject via a transurethral resection of bladder tumor. In some embodiments, the first biological sample is obtained or derived from the subject after performing a transurethral resection of bladder tumor. In some embodiments, the method comprises identifying a clinical intervention for the subject based at least in part on the detected presence or the absence of the cancer. In some embodiments, the clinical intervention is selected from a plurality of clinical interventions. In some embodiments, the clinical intervention is selected from the group consisting of: surgical resection, chemotherapy, radiotherapy, immunotherapy, adjuvant therapy, neoadjuvant therapy, androgen deprivation therapy, and a combination thereof. In some embodiments, the surgical resection is a transurethral resection of bladder tumor (TURBT) or a repeat transurethral resection of bladder tumor. In some embodiments, the method further comprises administering the clinical intervention to the subject. In some embodiments, the set of biomarkers comprises one or more members selected from the group consisting of genes listed in Table 1. In some embodiments, the set of biomarkers comprises 2, 3, 4, 5, 6, 7, 8, 9, 10, 15, 20, 25, 30, 35, 40, 45, 50, 55, 60, 65, 70, 75, 80, or 85 members selected from the group consisting of genes listed in Table 1. In some embodiments, the set of biomarkers comprises one or more members selected from the group consisting of genes listed in Table 2. In some embodiments, the set of biomarkers comprises 2, 3, 4, 5, 6, 7, 8, 9, 10, 15, 20, 25, 30, 35, or 40 members selected from the group consisting of genes listed in Table 3. In some embodiments, the set of biomarkers comprises one or more members selected from the group consisting of genes listed in Table 3. In some embodiments, the plurality of probes comprise nucleic acid primers. In some embodiments, the plurality of probes comprise nucleic acid capture probes. In some embodiments, the plurality of probes have sequence complementarity with at least a portion of nucleic acid sequences of the set of biomarkers. In some embodiments, the plurality of probes comprise at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 different probes. In some embodiments, the (d) further comprises sequencing using a fixed plurality of probes wherein the probes of the fixed plurality of probes comprises probes that do not comprise sequences of the subset of the set of biomarkers. In some embodiments, the fixed plurality of probes comprise one or more members selected from the group consisting of genes listed in Table 2. In some embodiments, the method further comprises determining a likelihood of the determination of the presence or the absence of the cancer in the subject. In some embodiments, the method further comprises monitoring the presence or the absence of the cancer in the subject, wherein the monitoring comprises assessing the presence or the absence of the cancer in the subject at each of a plurality of time points. In some embodiments, a difference in the assessment of the presence or the absence of the cancer in the subject among the plurality of time points is indicative of one or more clinical indications selected from the group consisting of: (i) a diagnosis of the cancer, (ii) a prognosis of the cancer, and (iii) an efficacy or non-efficacy of a course of treatment for treating the cancer of the subject. In some embodiments, the prognosis comprises an expected progression-free survival (PFS) or overall survival (OS).

In some embodiments, the set of biomarkers from the DNA molecules comprise tumor-associated alterations selected from the group consisting of: single nucleotide variants (SNVs), insertions or deletions (indels), and rearrangements. In some embodiments, the method further comprises determining, among the set of biomarkers, a mutant allele frequency of a set of somatic mutations. In some embodiments, the method further comprises determining a circulating tumor DNA (ctDNA) fraction of the cancer of the subject based at least in part on the set of mutant allele frequencies. In some embodiments, the method further comprises determining a tumor mutational burden (TMB) of the cancer of the subject. In some embodiments, the method further comprises determining an abnormality score of the cancer of the subject based at least in part on the set of mutant allele frequencies.

In another aspect, the present disclosure provides a method for providing a treatment for a subject, comprising: (a) assaying deoxyribonucleic acid (DNA) molecules from a tumor sample obtained or derived from the subject at a first time point, wherein the subject has undergone a transurethral resection of bladder tumor procedure; (b) detecting a set of biomarkers from the DNA molecules based at least in part on the assaying of (a), wherein the set of biomarkers comprise differentially expressed markers or variants; (c) generating a plurality of probe nucleic acids that are customized for the subject, wherein the probe nucleic acids comprises sequences of at least a subset of the set of biomarkers; (d) using the plurality of probe nucleic acids, sequencing DNA from a urine sample obtained or derived from the subject at a second time point to detect the presence or absence of the subset of the set of biomarkers, wherein the sequencing is performed at a depth of at least 80×, wherein the second biological sample is a urine sample; (e) computer processing the subset of the set of biomarkers to detect the presence or absence of minimal residual disease (MRD) in the subject; (f) based at least on the presence or absence of minimal residual disease (MRD) in the subject, performing a repeat a transurethral resection of bladder tumor (rTURBT) procedure.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows an example workflow.

FIG. 2 shows and oncoplot of urinary tumor DNA genomic alterations in patients with NMIBC before repeat TURBT.

FIG. 3A shows WES+Variant frequencies between index and rTURBT sample. FIG. 3B shows variant overlap panel between index TURBT (blue), rTURBT (green), and urine samples (yellow). FIG. 3C shows utDNA positivity in patients with disease present at rTURBT. FIG. 3D shows tumor fraction of utDNA prior to rTURBT.

FIG. 4 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Provided herein are systems and methods for detection of the presence or absence of cancer in a subject. The systems and methods provided herein comprises assaying polynucleotides to identify biomarkers of cancers in a subject. The biomarkers may be processed in order to identify the presence or absence of cancer. The methods described herein may process analytes to determine a presence or absence of cancer. The analytes may comprise cfDNA or other analytes that can be provided via non-invasive methods. By analyzing analytes obtained by non-invasive methods, the methods may allow for improved or similar detection or determination of a prognosis as compared to methods that use tumor or tissue biopsy.

Human urine can contain fragmented DNA known as urinary cell-free DNA (ucfDNA) that originates from dying cells in the urogenital tract or from circulating DNA passed through the glomerular filtration. Given the direct access of the urinary tract, urine is a viable source for detecting cfDNA biomarkers and ucfDNA may improve current diagnostic sensitivity for liquid biopsy in genitourinary cancers. Many gene variants can be identified in ucfDNA from cancer patients, particularly from bladder cancer patients. Urine can also contain whole cells, cell debris, and other biomolecules that may be pelleted via centrifugation and to form a pellet. The urine cell pellet (UCP) or urinary pellet may comprise DNA that may be analyzed for biomarkers. Using urine for liquid biopsy provides a completely noninvasive approach for detection of genomic biomarkers to guide cancer treatment.

Next-generation sequencing has revolutionized cancer genomic research in the last 10+ years. NGS technologies commercially available for guiding treatment plans for cancer patients that have been FDA cleared or approved for use in processing DNA from patient tissue or blood samples. A next-generation sequencing (NGS) assay on cfDNA can enable an accurate detection of genomic alterations, including single nucleotide variant (SNV), insertion and deletion (Indel), Copy number variation (CNV), and DNA re-arrangement. Urine cfDNA originates directly from dying cells exfoliated in urine and can be considered more representative of the tumor than a tissue biopsy due to tumor heterogeneity, since tissue biopsy can only account for mutations found in a specific region of the tumor. In addition, urine can contain fewer contaminating proteins than blood, and the cfDNA level in urine may be greater than in the bloodstream. Urinary cfDNA can be subjected to sequencing to allow detection of cancer and genetic alterations associated with cancer from a subject's urine. Methods and assays described in this disclosure represents an application of the NGS technology and provides a non-invasive, cost-effective, and potentially more sensitive sampling method for patients with cancer, such as genitourinary cancers, including bladder cancer.

In addition to staging and grading of a patient's tumor, tissue biopsy often represent the gold standard in guiding treatment for cancer patients. However, depending on the location of the tumor or the condition of the patient, tumor biopsies can be painful, and the patient may incur risk of complication, whereby medical treatment can become costly. In some cases, tissue biopsy may not be feasible. Less invasive sampling methods remain an unmet clinical need for treatment of bladder cancer patients. A urine cfDNA Assay and the option for liquid biopsy (e.g., from urine) may help fill the void. In addition, there is opportunity for patients to be tested multiple times by urine liquid biopsy as opposed to tissue biopsy. Therefore, liquid biopsy from urine represents a non-invasive and cost-effective method for obtaining patient samples to determine molecular eligibility for certain treatment strategies.

Urine liquid biopsy samples can also improve patient care. In instances where tissue biopsy is not feasible for bladder cancer patient NGS testing, bladder cancer patients can be monitored using urine liquid biopsy. The assays can detect other gene mutations in urine samples from bladder cancer patients, including but not limited to alterations in CDKN2A, HRAS/KRAS, KDM6A, PIK3CA, TERT, TP53 and TSC1, which if identified may help to inform patient treatment.

Bladder cancer is the tenth most common malignancy worldwide, with an estimated 550,000 new cases and 200,000 deaths reported in 2018. The majority of bladder cancer cases are non-muscle invasive bladder cancer (NMIBC), requiring intensive regimens of frequent monitoring, local resection (transurethral resection of bladder tumor [TURBT]), and intravesical therapies to reduce the risk of both recurrent and progressive disease. Despite these efforts, within 5 years 45% to 60% of patients will experience recurrent disease, and nearly 20% of those with high-risk disease will progress to muscle-invasive tumors requiring radical cystectomy (RC). The natural history of high-risk NMIBC is unpredictable; rates of recurrence vary from 15% to 78% and rates of progression to muscle invasion and metastasis vary from <1% to 45%. Long-term outcomes suggest approximately 20% to 25% of the high-risk NMIBC patients ultimately die from bladder cancer.

TURBT may be effective as a therapy for the treatment of cancer. Even with complete removal of a tumor with TURBT, recurrence of cancer can still occur. As such, there is a need for monitoring patients that have undergone TURBT to determine if they need additional therapeutic intervention. Specifically, a repeat TURBT (rTURBT) may be performed on the subject. The methods described herein may allow for monitoring of a patient after an initial TURBT. Based on the monitoring and MRD, another therapeutic intervention such as a rTURBT may be recommended or performed on the subject.

Analytes that can be used for tumor diagnosis from urine liquid biopsy include cfDNA, non-coding-RNA, exfoliated tumor cells and proteins. During tumor destruction therapies or during apoptotic and necrotic processes, both healthy and diseased cells may release cfDNA fragments that are typically 100-200 base pairs in length. In patients without disease, the phagocytic cells englobe cellular debris and necrotic cells, and thus there are very low levels of cfDNA. In the case of patients with disease, phagocytosis is compromised, DNA digestion is minimal, and the DNA fragments have a random dimension that could exceed 10,000 base pairs. Therefore, the cfDNA level in a patient with disease is often elevated.

Urine cfDNA (ucfDNA) can be extracted from a urine from a subject and ucfDNA can be subjected to various reactions to allow for sequencing of the ucfDNA. Library construction of ucfDNA can comprise amplification, ligation of adapter or additional sequences and/or labeling with barcodes to generate a sequencing library. Additionally, a ucfDNA or ucfDNA library can be subjected to enrichment using capture probes or amplification primers to enrich for specific sequences of interest from the cfDNA. The library can then be subjected to sequencing reactions to generate sequencing data.

A urine sample may be isolated and collected from an individual. Following collection, extraction of urinary DNA or cfDNA can performed. Library construction can then be performed on the extracted urinary DNA or cfDNA followed by enrichment of specific targets. Once enrichment is performed, the urinary DNA or cfDNA can be sequenced and the sequencing data can then be processed.

Genetic alterations, such as single nucleotide variations (SNVs), indels, DNA rearrangements, and copy number variations (CNVs) can be identified by bioinformatic analysis of the sequencing data. Generally, a bioinformatic pipeline can utilize the raw sequencing data (e.g., BCL files) and output mutational calls. The pipeline can perform various tasks to analyze the sequencing data such as adapter trimming, barcode checking, or error correction. Cleaned paired files (e.g. FASTQ files) can be aligned to human reference genome using an alignment tool such as BWA alignment tool. Consensus sequences can then be derived by merging paired-end reads that originated from the same molecules as single strand fragments. Single strand fragments from the same double strand DNA molecules can be further merged as double stranded. These processes can allow for sequencing and PCR errors to be corrected.

The subject may be a suspected of a suffering from a cancer. The cancer may be specific or originating from an organ or other area of the subject. For example, the cancer may be breast cancer, lung cancer, prostate cancer, colorectal cancer, melanoma, bladder cancer, non-Hodgkin lymphoma, kidney cancer, endometrial cancer, leukemia, pancreatic cancer, thyroid cancer, and liver cancer, and any combination thereof. The cancer may be a hormone sensitive prostate cancer (HSPC), castrate-resistant prostate cancer (CRPC), metastatic prostate cancer, and a combination thereof. The cancer may be a cancer of a tissue or cell of the genitourinary tract. For example, the cancer may be a bladder cancer, kidney cancer, or prostate cancer. The cancer may comprise biomarkers that are specific to a particular cancer. The specific biomarkers may indicate a presence of a particular cancer. For example, biomarker may indicate that a castrate-resistant prostate cancer is present. The identification of the presence of a type of cancer may allow the determination of a treatment option or recommendation.

In some cases, the subject may be asymptomatic for cancer. For example, the cancer may not exhibit any symptoms and the subject may be unaware of the presence of cancer. The methods described herein may allow a cancer to be identified at an earlier stage than otherwise. The identification of the presence of the cancer at an earlier stage may allow a treatment option or recommendation to be determined at an earlier stage and may allow the subject to have an improved prognosis.

The biological sample may comprise nucleic acids. The biological sample be a cell-free deoxyribonucleic acid (cfDNA) sample or a cell-free ribonucleic acid (cfRNA) sample. The biological sample may comprise genomic DNA or germline DNA (gDNA). The nucleic acid may be a DNA (e.g. double-stranded DNA, single-stranded DNA, single-stranded DNA hairpins, cDNA, genomic DNA, germline DNA, circulating tumor DNA (ctDNA), cell-free DNA (cfDNA)), an RNA (e.g. cfRNA, mRNA, CRNA, miRNA, siRNA, miRNA, snoRNA, piRNA, tiRNA, snRNA), or a DNA/RNA hybrids. The biological sample may be a derived from or contain a biological fluid. For example, the biological sample may be a plasma sample, a serum sample, a buffy coat sample, a peripheral blood mononuclear cell (PBMC) sample, a red blood cell sample, a urine sample, a saliva sample, or other body fluid sample. The biological sample may comprise or be a pleural fluid sample, peritoneal fluid sample, amniotic fluid sample, cerebrospinal fluid sample, lymphatic fluid sample, sweat sample, tear sample, semen sample, or any combination of biological fluid. The biological sample may comprise a urine sample.

The biological sample may be collected, obtained, or derived from the subject using a collection tube. The collection tube may be an ethylenediaminetetraacetic acid (EDTA) collection tube, a cell-free RNA collection tube, or a cell-free deoxyribonucleic acid (DNA) collection tube and CTC collection tubes, or other blood collection tube. The collection tube may comprise additional reagents for stabilizing the nucleic acid molecules or blood cells. The collection tube may allow the nucleic acid or blood cells to be stable such to minimize degradation of the biological sample prior to assaying. The additional reagents may comprise buffer salts or chelators.

The biological sample may be obtained or derived from a subject at various times. The biological sample may be obtained or derived from a subject prior to the subject receiving a therapy for cancer. The biological sample may be obtained or derived from a subject during receiving a therapy for cancer. The biological sample may be obtained or derived from a subject after receiving a therapy for cancer. The biological sample may be obtained or derived from said subject via a transurethral resection of bladder tumor. The biological sample may be obtained or derived from said subject after performing a transurethral resection of bladder tumor.

The biological sample may be collected over 1, 2, 3, 4, 5, 6, 7, 8, 9, 10, 11, 12, 15, 20, 30, 40, 50, 60, 70, 80, 90, 100, 200, 300, 400, 500, 600, 700, 800, 900, 1000 or time points. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more hour period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more day period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more week period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more month period. The time points may occur over a 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60 or more year period.

In various aspects as described herein, a clinical intervention or a therapy may be identified at least in part based on the identification of the presences of cancer, or the presence of a parameter of cancer. The clinical intervention may be a plurality of clinical interventions. The clinical intervention may be selected from a plurality of clinical interventions. The clinical intervention may be a surgical resection, chemotherapy, radiotherapy, immunotherapy, adjuvant therapy, neoadjuvant therapy, androgen deprivation therapy, or a combination thereof. The clinical intervention may be a transurethral resection of bladder tumor (TURBT). The clinical intervention may be a repeat transurethral resection of bladder tumor (rTURBT). In some cases, the clinical interventions may be administered to the subject. After administration of the clinical intervention, a sample may be obtained or derived from the subject such to monitor the cancer or cancer parameters. As such, the methods and systems disclosed herein may be performed iteratively such that monitoring of a cancer can be performed. Additionally, by performing the methods or systems iteratively, therapies or clinical interventions may be updated based on the results of the methods. The monitoring of the cancer may include an assessment as well as a difference in assessment from a previously generated assessment. The difference in an assessment of cancer in the subject among a plurality of time points (or samples) may be indicative of one or more clinical indications such as a diagnosis of the cancer, a prognosis of the cancer, or an efficacy or non-efficacy of a course of treatment for treating the cancer of the subject. The prognosis may comprise expected progression-free survival (PFS), overall survival (OS), or other metrics relating the severity or survivability of a cancer.

The biological samples may be subjected to additional reactions or conditions prior to assaying. For example, the biological sample may be subjected to conditions that are sufficient to isolate, enrich, or extract nucleic acids, such cfDNA molecules.

The methods disclosed herein may comprise conducting one or more enrichment reactions on one or more nucleic acid molecules in a sample. The enrichment reactions may comprise contacting a sample with one or more beads or bead sets. The enrichment reactions may comprise one or more hybridization reactions. For example, the enrichment reactions may comprise contacting a sample with one or more capture probes or bait molecules that hybridize to a nucleic acid molecule of the biological sample. The enrichment reaction may comprise differential amplification of a set of nucleic acid molecules. The enrichment reaction may enrich for a plurality of genetic loci or sequences corresponding to genetic loci. The enrichment reactions may comprise the use of primers or probes that may complementarity to sequences (or sequences upstream or downstream) of a sequence that is to be enriched. For example, a capture probe may comprise sequence complementarity to a set of genomic loci and allow the enrichment of the genomic loci. The enrichments reactions may comprise a plurality of probes or primers. For example, a capture probe may comprise sequence complementarity to a gene selected from Table 1, Table 2, or Table 3. A plurality of probes may comprise 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, or 600 different probes.

The methods disclosed herein may comprise conducting one or more isolation or purification reactions on one or more nucleic acid molecules in a sample. The isolation or purification reactions may comprise contacting a sample with one or more beads or bead sets. The isolation or purification reaction may comprise one or more hybridization reactions, enrichment reactions, amplification reactions, sequencing reactions, or a combination thereof. The isolation or purification reaction may comprise the use of one or more separators. The one or more separators may comprise a magnetic separator. The isolation or purification reaction may comprise separating bead bound nucleic acid molecules from bead free nucleic acid molecules. The isolation or purification reaction may comprise separating capture probe hybridized nucleic acid molecules from capture probe free nucleic acid molecules. The isolation reactions may comprises removing or separating a group of nucleic acid molecules from another group of nucleic acids.

The methods disclosed herein may comprise conduction extraction reactions on one or more nucleic acids in a biological sample. The extraction reactions may lyse cells or disrupt nucleic acid interactions with the cell such that the nucleic acids may be isolated, purified, enriched, or subjected to other reactions.

The methods disclosed herein may comprise amplification or extension reactions. The amplification reactions may comprise polymerase chain reaction. The amplification reaction may comprise PCR-based amplifications, non-PCR based amplifications, or a combination thereof. The one or more PCR-based amplifications may comprise PCR, qPCR, nested PCR, linear amplification, or a combination thereof. The one or more non-PCR based amplifications may comprise multiple displacement amplification (MDA), transcription-mediated amplification (TMA), nucleic acid sequence-based amplification (NASBA), strand displacement amplification (SDA), real-time SDA, rolling circle amplification, circle-to-circle amplification or a combination thereof. The amplification reactions may comprise an isothermal amplification.

The method disclosed herein may comprise a barcoding reaction. A barcoding reaction may comprise the additional of a barcode or tag to the nucleic acid. The barcode may be a molecular barcode or a sample barcode. For example, a barcode nucleic acid may comprise a barcode sequence which may be a degenerate n-mer. The sequence may be randomly generated or generated such to synthesize a specific barcode sequence. The barcode nucleic acid may be added to a sample such to label the nucleic acid molecules in the sample. The barcodes may be specific to a sample. For example, a plurality of barcode nucleic acids may be added to a sample in which the barcode sequence is the same. Upon barcoding of the nucleic acids, those originating from a same sample may have a same barcode sequence, and may allow a nucleic acid to be identified as belonging to a particular or given sample. A molecular barcode may also be used such that each molecule (or a plurality of molecules) in a same volume have a different molecular barcode. This barcode may be subjected to amplification such that all amplicons derived from a molecule have the same barcode. In this way, molecules originating from a same molecule may be identified. The sequences reads may be processed based on the barcode sequences. For example, the processing may reduce errors or allow a molecule to be tracked. Barcode sequences may be appended or otherwise added or incorporated into a sequence by various reactions, for example an amplification, extension, or ligation reaction, and may be performed enzymatically using a nucleic acid polymerase or ligase. The ligation may be an overhang or blunt end ligation and the barcodes may comprise complementarity to nucleic acids to be barcoded. This complementarity may be a sequence derived from the sample from the subject or may be constant sequence generated via a reaction performed on the nucleic acids in the sample.

In some cases, the biological sample may comprise multiple components. For example, the biological sample may be a whole blood sample. The biological sample may be subjected to reactions such to separate or fractionate a biological sample. For example, a whole blood sample may be a fractionated and cell free nucleic acids may be obtained. The whole blood sample may be fractionated using centrifugation such that blood cells may be separated from the plasma (which may contain cell free nucleic acid). A sample may be subjected to multiple rounds of separation or fractionation.

In various aspects described throughout the disclosure, the nucleic acids may be subjected to sequencing reactions. The sequencing the reactions may be used on DNA, RNA or other nucleic acid molecules. Example of a sequencing reaction that may be used include capillary sequencing, next generation sequencing, Sanger sequencing, sequencing by synthesis, single molecule nanopore sequencing, sequencing by ligation, sequencing by hybridization, sequencing by nanopore current restriction, or a combination thereof. Sequencing by synthesis may comprise reversible terminator sequencing, processive single molecule sequencing, sequential nucleotide flow sequencing, or a combination thereof. Sequential nucleotide flow sequencing may comprise pyrosequencing, pH-mediated sequencing, semiconductor sequencing or a combination thereof. The sequencing reactions may comprise whole genome sequencing, whole exome sequencing, low-pass whole genome sequencing, targeted sequencing, methylation-aware sequencing, enzymatic methylation sequencing, bisulfite methylation sequencing. The sequencing reaction may be a transcriptome sequencing, mRNA-seq, totalRNA-seq, smallRNA-seq, exosome sequencing, or combinations thereof. Combinations of sequencing reactions may be used in the methods described elsewhere herein. For example, a sample may be subjected to whole genome sequencing and whole transcriptome sequencing. As the samples may comprise multiple types of nucleic acids (e.g. RNA and DNA), sequencing reactions specific to DNA or RNA may be used such to obtain sequence reads relating to the nucleic acid type.

The sequencing reactions can be performed at various sequencing depths. The sequencing depths of a sequencing reaction may be selected or modulated. The sequencing reactions may comprise sequencing at a region a depth of at least 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 90,000, 100,000×, or more. The sequencing reactions may comprise sequencing a region at a depth of no more than 1×, 2×, 3×, 4×, 5×, 6×, 7×, 8×, 9×, 10×, 11×, 12×, 13×, 14×, 15×, 16×, 17×, 18×, 19×, 20×, 25×, 30×, 35×, 40×, 45×, 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 90,000, 100,000×, or less.

In various embodiments, a low pass whole genome sequencing is used to sequence nucleic acids. The low pass whole genome sequence may be performed at an average sequencing depth of at least 1×, 2×, 3×, 4×, 5×, 6×, 7×, 8×, 9×, 10×, or more. The low pass whole genome sequence may be performed at an average sequencing depth of no more than 1×, 2×, 3×, 4×, 5×, 6×, 7×, 8×, 9×, 10×, or less. The low pass whole genome sequencing may be performed at an average depth of between 1× and 2×.

In various embodiments, a sequencing reaction may be performed using a set of personalized or customized probes. The sequencing reaction using a set of personalized or customized probes may be a deep sequencing reaction or ultra-deep sequencing reaction. For example, the sequencing reaction using a set of personalized or customized probes may be performed at an sequencing depth of 50×, 60×, 70×, 80×, 90×, 100×, 200×, 300×, 400×, 500×, 600×, 700×, 800×, 900×, 1000×, 2000×, 3000×, 4000×, 5000×, 6000×, 7000×, 8000×, 9000×, 10,000×, 20,000×, 30,000×, 40,000×, 50,000×, 60,000×, 70,000×, 80,000×, 90,000, 100,000×, or more.

In various embodiments, a whole exome sequencing is used to sequence nucleic acids of a subject. The whole exome sequencing may be performed at a non-uniform depth. For example, certain areas of the exome may be boosted or otherwise sequenced at a greater depth than other regions, or at a greater depth than the average depth of the whole exome sequencing. By sequencing certain regions at a higher depth, genes or regions that are of more interest may be analyzed with higher sensitivity, accuracy, and/or precision. Genes or regions associated with or related to cancer can be sequenced at a greater depth. For example, at least 100, 200, 300, 400, 500, 600, 700, 800, 900, or more genes can be sequenced at a higher depth than the rest of the exome (e.g. average depth of the whole exome sequencing).

The sequencing of nucleic acids may generate sequencing read data. The sequencing reads may be processed such to generate data of improved quality. The sequencing reads may be generated with a quality score. The quality score may indicate an accuracy of a sequence read or a level or signal above a nose threshold for a given base call. The quality scores may be used for filtering sequencing reads. For example, sequencing reads may be removed that do not meet a particular quality score threshold. The sequencing reads may be processed such to generate a consensus sequence or consensus base call. A given nucleic acid (or nucleic acid fragment) may be sequenced and errors in the sequence may be generated due to reactions prior or during sequencing. For example, amplification or PCR may generate error in amplicons such that the sequences are not identical to a parent sequence. Using sample barcodes or molecular barcodes, error correction may be performed. Error correction may include identifying sequence reads that do not corroborate with other sequences from a same sample or same original parent molecules. The use of barcodes may allow the identification or a same parent or sample. Additionally, the sequence reads may be processed by performing single strand consensus calling or double stranded consensus call, thereby reducing or suppressing error.

The methods as disclosed herein may comprise determining allele frequency or other cancer related metric. The methods may comprise a mutant allele frequency of a set of somatic mutation among a set of biomarkers. The mutant allele frequency may be used to determine a circulating tumor DNA (ctDNA) fraction of a cancer of a subject. A plasma tumor mutational burden (pTMB) of a cancer of the subject may be determined based at least in part on the set of mutant allele frequencies. Detection of microsatellite instability may also be used to determine the presence or absence of a cancer or cancer metric. Methylation states may be determined using methods described herein and may be used to identify a presence of a cancer or cancer parameter.

In various aspects, sets of biomarkers are processed and data corresponding to the biomarkers are generated. The sets of biomarkers may comprise quantitative measures from a set of cancer-associated genomic loci. The cancer-associated genomic loci may correspond to a set of genes. The cancer associated genomic loci may comprise one or more genes selected from Table 1. In some case, a set of cancer associated genomic loci comprises 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, or 180 members selected from the group consisting of genes listed in Table 1.

TABLE 1
List of genes

| | | | | | | | |
|---|---|---|---|---|---|---|---|
| ABRAXAS1 | AKT1 | ALK | APC | AR | ARAF | ARID1A | ATM |
| ATR | BAP1 | BARD1 | BRAF | BRCA1 | BRCA2 | BRIP1 | CCND1 |
| CCNE1 | CD274 | CDH1 | CDK12 | CDK4 | CDK6 | CDKN2A | CHEK1 |
| CHEK2 | CTNNB1 | DDR2 | DNAJB1 | EGFR | EPCAM | ERBB2 | ERBB3 |
| ERCC1 | ERCC2 | ERCC4 | ESR1 | EZH2 | FANCA | FANCC | FANCD2 |
| FANCI | FANCL | FANCM | FBXW7 | FGFR1 | FGFR2 | FGFR3 | GNA11 |
| GNAQ | GNAS | HDAC2 | HRAS | IDH1 | IDH2 | JAK2 | JAK3 |
| KIT | KRAS | MAP2K1 | MAP2K2 | MAPK1 | MDM2 | MET | MLH1 |
| MPL | MRE11 | MSH2 | MSH6 | MTOR | MYC | MYCN | MYD88 |
| NBN | NF1 | NFE2L2 | NPM1 | NRAS | NTRK1 | NTRK3 | PALB2 |
| PDCD1LG2 | PDGFRA | PIK3CA | PMS2 | POLD1 | POLE | PPM1D | PPP2R1A |
| PPP2R2A | PRKACA | PRKD1 | PTEN | PTPN11 | RAD50 | RAD51 | RAD51B |
| RAD51C | RAD51D | RAD54L | RAF1 | RB1 | RECQL | RET | RNF43 |
| ROS1 | RPA1 | SMAD4 | SMO | SPOP | STK11 | TERT | TMPRSS2 |
| TP53 | TP53BP1 | TSC1 | TSC2 | VHL | XRCC2 | XRCC3 | XRCC4 |

The sets of biomarkers may comprise one or more genes selected from Table 2 In some case, a set of biomarkers may comprise 2, 3, 4, 5, 6, 7, 8, 9, 10, 15, 20, 25, 30, 35, 40, 45, 50, 55, 60, 65, 70, 75, 80, or 85 members selected from the group consisting of genes listed in Table 2.

The sets of biomarkers may comprise one or more genes selected from Table 3. In some case, a set of biomarkers may comprise 2, 3, 4, 5, 6, 7, 8, 9, 10, 15, 20, 25, 30, 35, or 40 members selected from the group consisting of genes listed in Table 3.

The sets of biomarkers may correspond to genetic aberration of a genetic locus. The genetic aberration may a tumor associated alteration. The genetic aberration may comprise copy number alterations (CNAs), copy number losses (CNLs), single nucleotide variants (SNVs), insertions or deletions (indels), and/or rearrangements. The set of biomarkers may be identified in a variety of nucleic acid types. For example, the tumor associated alteration may be identified in cfDNA. The tumor associated alteration may comprise changes in allelic expression, or gene expression. Methods and systems disclosed herein may allow for gene expression profiling and identification of changes to the expression levels of gene.

In various aspects, the methods may comprise identifying the presence of a cancer or a cancer parameter. The methods may comprises determining a probability or a likelihood of the presence of cancer or a cancer parameter. For example, instead of a binary output indicating a presence or absence, an output may be generated that indicates a probability that subject has cancer. This probability may be determined based on algorithms as described elsewhere herein. Similarly, a probability or likely of response to a particular treatment or a probability of relapse may be outputted.

In various aspects, the sets of biomarkers are processed using an algorithm. The algorithm may be a trained algorithm. The trained algorithms may use the sets of biomarkers as an input and generate an output regarding the presence or absence of a cancer. The output may be specific to a type of cancer or subtype of cancer. For example, the output may indicate the presence of bladder cancer.

The trained algorithm may be trained on multiple samples. For example, the trained algorithm may be trained using at least 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or more independent training samples. The trained algorithm may be trained using no more 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 300, 400, 500, 600,700, 800, 900, 1000, 1100, 1200, 1300, 1400, 1500, 1600, 1700, 1800, 1900, 2000, 3000, 4000, 5000, 6000, 7000, 8000, 9000, 10000, or less, independent training samples. The training samples may be associated with a presence or an absence of the cancer. The training samples may be associated with a relapse of cancer. The training samples may be associated with cancer that is resistant to a particular drug or treatment. An individual training sample may be positive for a particular cancer. An individual training sample may be negative for a particular cancer. By using training samples, the trained algorithm may be able to detect a cancer, determine a probability of recurrence or relapse of a cancer, or determine if a cancer comprises a set of biomarkers may be resistant to a treatment. The training sample may be associated with additional clinical health data of a subject. For example, additional clinical health data may comprise the gender, weight, height, or levels of metabolites or antibodies in a subjects. Additional clinical health data may comprise indication of other diseases, disorders, or diseases conditions.

The trained algorithms may be trained using multiple sets of training samples. The sets may comprise training samples as described elsewhere herein. For example, the training may be performed using a first set of independent training samples associated with a presence of the cancer and a second set of independent training samples associated with an absence of the cancer. Similarly, a first set may be associated with relapse and a second sample may be associated with the absence of relapse.

The trained algorithm may also process additional clinical health data of the subject. For example, additional clinical health data may comprise the gender, weight, height, or levels of metabolites or antibodies in a subject. Additional clinical health data may comprise indication of other diseases, disorders, or diseases conditions that the subject may suffer from. By using the additional clinical health data, in conjunction with the biomarkers, the trained algorithm may output a presence or absences of cancer, probability of relapse, or resistance to drug treatment, which may be different from the output of an algorithm that does not process additional clinical health.

The trained algorithm may be an unsupervised machine learning algorithm. For example, the unsupervised machine learning algorithm may utilize cluster analysis to identify attributes of interest. The trained algorithm may be a supervised machine learning algorithm. For example, the trained algorithm may be trained with training data such to generate an expected or desired output. The supervised learning algorithm may comprise a deep learning algorithm, a support vector machine (SVM), a neural network, or a Random Forest. Via the machine learning algorithm, the trained algorithm may be able to identify relationships of biomarkers to a particular cancer prognosis or diagnosis. Without the trained algorithm, it may otherwise be difficult to identify relationships of the biomarkers to accurately identify the presence of a cancer or other parameters associated with the cancer.

In various aspects, the systems and methods may comprise an accuracy, sensitivity, or specificity of detection of the cancer or a parameter of the cancer. For example, the methods or systems may comprise detecting the presence or the absence of cancer (or the presence of a parameter of the cancer, such as recurrence, relapse, or drug resistance) in the subject at an accuracy of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a sensitivity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a specificity of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a positive predictive value of at least about 60%, at least about 70%, at least about 75%, at least about 80%, at least about 85%, at least about 90%, at least about 95%, at least about 98%, or at least about 99%. The methods or systems may comprise detecting the presence or the absence of cancer (or the presence of a parameter of the cancer, such as recurrence, relapse, or drug resistance) in the subject at a negative predictive value of at least about 60%, at least about 70%, at least about 75%, at least about 80%, at least about 85%, at least about 90%, at least about 95%, at least about 98%, or at least about 99%.

### Computer Control Systems

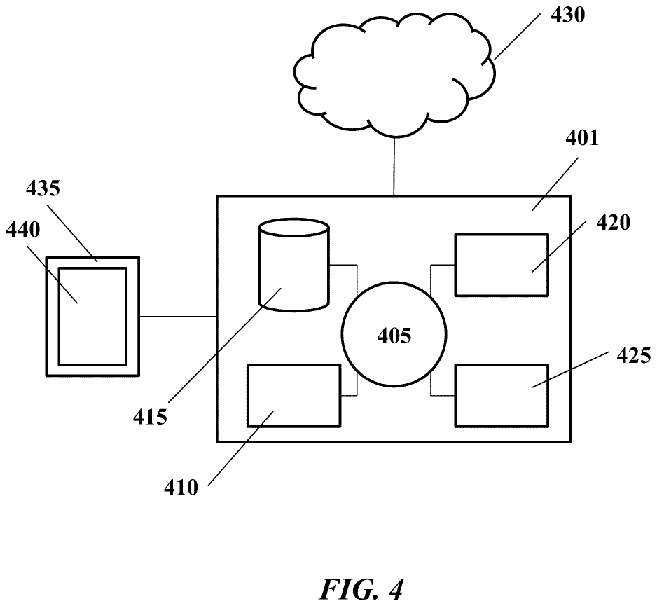

The present disclosure provides computer systems that are programmed to implement methods of the disclosure. FIG. 4 shows a computer system 401 that is programmed or otherwise configured to perform analysis or operations of the methods, for example determine a likelihood of the presence of a cancer based on a set of biomarkers of an individual or run an algorithm. The computer system 401 can regulate various aspects of methods and systems of the present disclosure, such as, for example, perform an algorithm, input training data, analyze sets of biomarker, or output a result for the user as to the presence or absence of cancer. The computer system 401 can be an electronic device of a user or a computer system that is remotely located with respect to the electronic device. The electronic device can be a mobile electronic device.

The computer system 401 includes a central processing unit (CPU, also “processor” and “computer processor” herein) 405, which can be a single core or multi core processor, or a plurality of processors for parallel processing. The computer system 401 also includes memory or memory location 410 (e.g., random-access memory, read-only memory, flash memory), electronic storage unit 415 (e.g., hard disk), communication interface 420 (e.g., network adapter) for communicating with one or more other systems, and peripheral devices 425, such as cache, other memory, data storage and/or electronic display adapters. The memory 410, storage unit 415, interface 420 and peripheral devices 425 are in communication with the CPU 405 through a communication bus (solid lines), such as a motherboard. The storage unit 415 can be a data storage unit (or data repository) for storing data. The computer system 401 can be operatively coupled to a computer network (“network”) 430 with the aid of the communication interface 420. The network 430 can be the Internet, an internet and/or extranet, or an intranet and/or extranet that is in communication with the Internet. The network 430 in some cases is a telecommunication and/or data network. The network 430 can include one or more computer servers, which can enable distributed computing, such as cloud computing. The network 430, in some cases with the aid of the computer system 401, can implement a peer-to-peer network, which may enable devices coupled to the computer system 401 to behave as a client or a server.

The CPU 405 can execute a sequence of machine-readable instructions, which can be embodied in a program or software. The instructions may be stored in a memory location, such as the memory 410. The instructions can be directed to the CPU 405, which can subsequently program or otherwise configure the CPU 405 to implement methods of the present disclosure. Examples of operations performed by the CPU 405 can include fetch, decode, execute, and writeback.

The CPU 405 can be part of a circuit, such as an integrated circuit. One or more other components of the system 401 can be included in the circuit. In some cases, the circuit is an application specific integrated circuit (ASIC).

The storage unit 415 can store files, such as drivers, libraries and saved programs. The storage unit 415 can store user data, e.g., user preferences and user programs. The computer system 401 in some cases can include one or more additional data storage units that are external to the computer system 401, such as located on a remote server that is in communication with the computer system 401 through an intranet or the Internet.

The computer system 401 can communicate with one or more remote computer systems through the network 430. For instance, the computer system 401 can communicate with a remote computer system of a user (e.g., a medical professional or patient). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 401 via the network 430.

Methods as described herein can be implemented by way of machine (e.g., computer processor) executable code stored on an electronic storage location of the computer system 401, such as, for example, on the memory 410 or electronic storage unit 415. The machine executable or machine readable code can be provided in the form of software. During use, the code can be executed by the processor 405. In some cases, the code can be retrieved from the storage unit 415 and stored on the memory 410 for ready access by the processor 405. In some situations, the electronic storage unit 415 can be precluded, and machine-executable instructions are stored on memory 410.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 401, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 401 can include or be in communication with an electronic display 435 that comprises a user interface (UI) 440 for providing, for example, an input of biomarkers or sequencing data, or an visual output relating to a detection, diagnosis, or prognosis. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 405. The algorithm can, for example, determine a presence or absence of a cancer or cancer parameter based on a set of input sequencing data from a sample derived from a subject.

### EXAMPLES

### Example 1 Minimal Residual Disease Detection Prior to Repeat Transurethral Resection of Bladder Tumor (TURBT)

Cell-free urinary tumor DNA (utDNA) generated from tumor genomic sequencing is an emerging biomarker showing tremendous potential for detecting minimal residual disease (MRD) in muscle-invasive and metastatic bladder cancer. A study was performed to assess utDNA's ability to measure MRD at the time of standard-of-care repeat transurethral resection of bladder tumor (rTURBT) in non-muscle invasive bladder cancer (NMIBC).

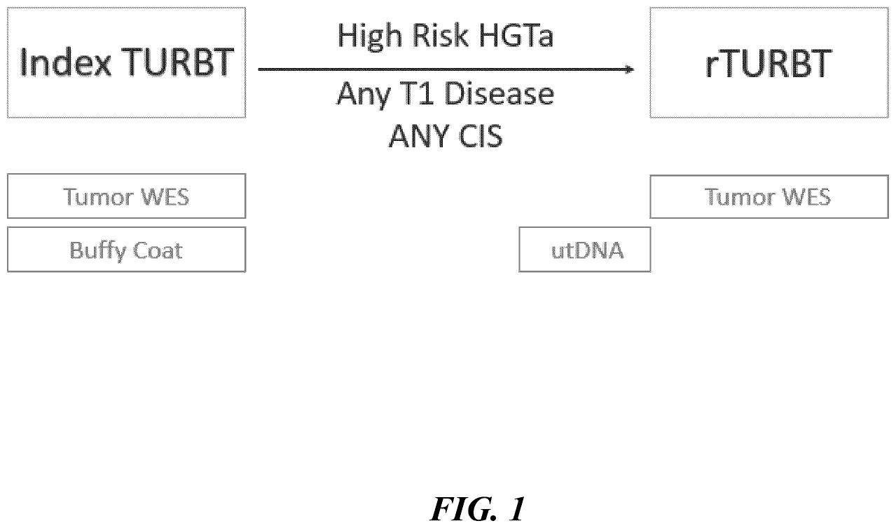

FIG. 1 shows a schematic of the study. An initial sample is taken from a subject as an index sample. This initial sample can be prior to any transurethral resection of bladder tumor (TURBT) or after an initial TURBT has been performed. Generally, rTURBT may be performed on subjects that show high risk tumors such as HGTa (high grade Ta tumors), and T1 tumors or and CIS (carcinoma in-situ) grade tumors. Patients with high-risk NMIBC were enrolled prior to rTURBT. Index tumor and rTURBT mutational profile was performed via whole exome sequencing (e.g., PredicineWES) across 20,000 genes. Urine samples (or urine cell pellet samples) were taken immediately prior to the repeat TURBT. utDNA detection was performed by ultra-deep sequencing of urinary cfDNA using a custom panel of up to 50-baseline mutations per patient together with a fixed core panel covering hotspot regions and actionable variants (e.g., using PredicineBEACON, Table 2). Urinary tumor fraction was used to call utDNA positivity. The primary endpoint was the detection of utDNA to predict MRD at the time of repeat-TURBT.

TABLE 2
List of genes with detected by the fixed actionable/hotspot panel sequencing.

ACSF3, AHR, ALDH3A2, ANAPC1, ANK2, ANKHD1, AR, ARID1A, ARID1B, ATM,
BLM, BRAF, BRCA1, BRCA2, C17orf97, C3orf70, CASP8, CDKN1A, CDKN2A, CER1,
CREBBP, CTNNB1, CYC1, DIDO1, DNMT3A, EGFR, ELF3, EP300, EPHB1, EPYC, ERBB2,
ERBB3, ERCC2, ESPL1, FBN3, FBXW7, FGFR1, FGFR2, FGFR3, FGFR4, FMN2, FOXA1,
HELZ, HIRIP3, HIST1H1C, HRAS, HRH4, IKZF1, KANSL1, KDM6A, KMT2A, KMT2D,
KRAS, MROH2B, MYC, MYH9, NF1, NFE2L2, NTRK1, NTRK3, PIAS1, PIK3CA, PKHD1,
PTEN, PTPRT, RARS2, RB1, RHOA, RHOB, RXRA, SACS, SF3B1, SF3B3, SOX2, SPOP,
STAG2, SYNE1, TEKT1, TERT, TMEM132D, TNKS, TP53, TSC1, VHL, WDR66, ZFP36L1

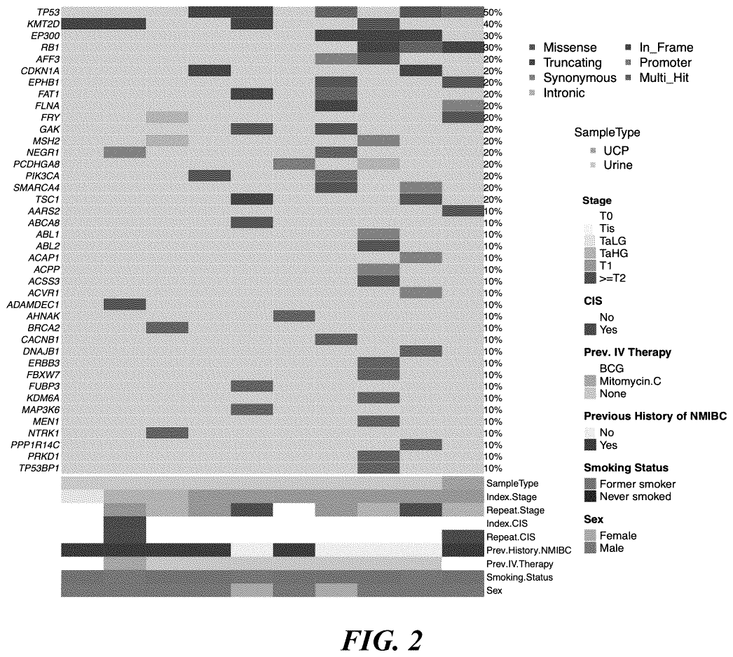

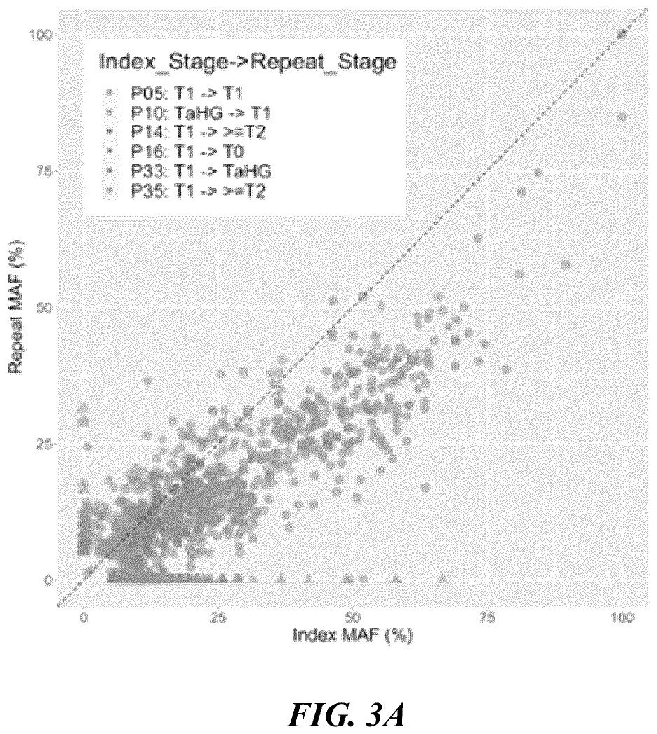

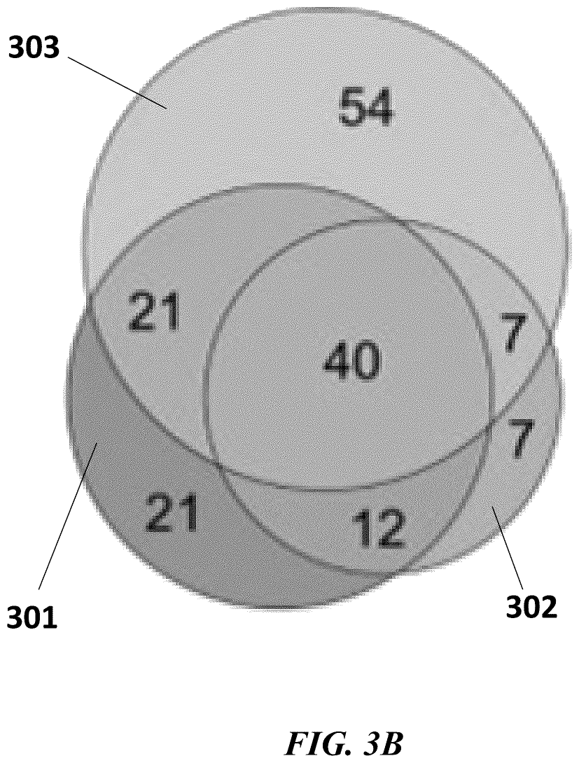

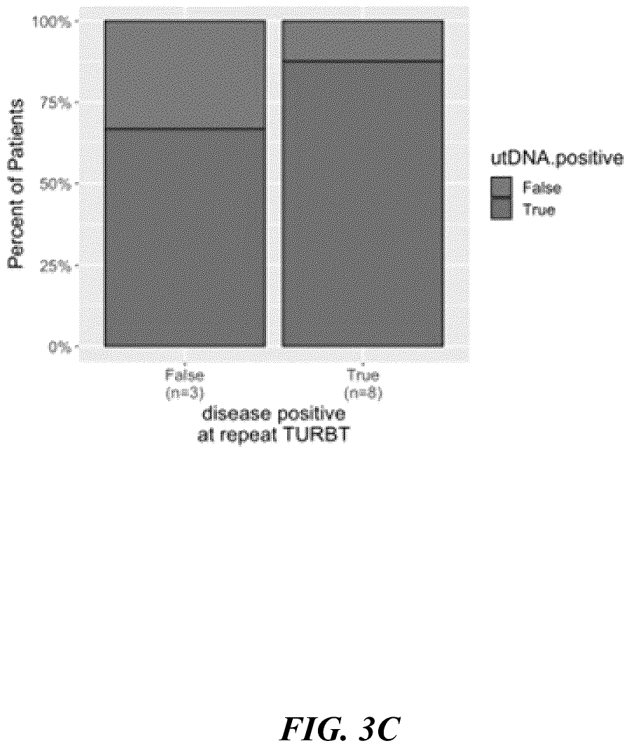

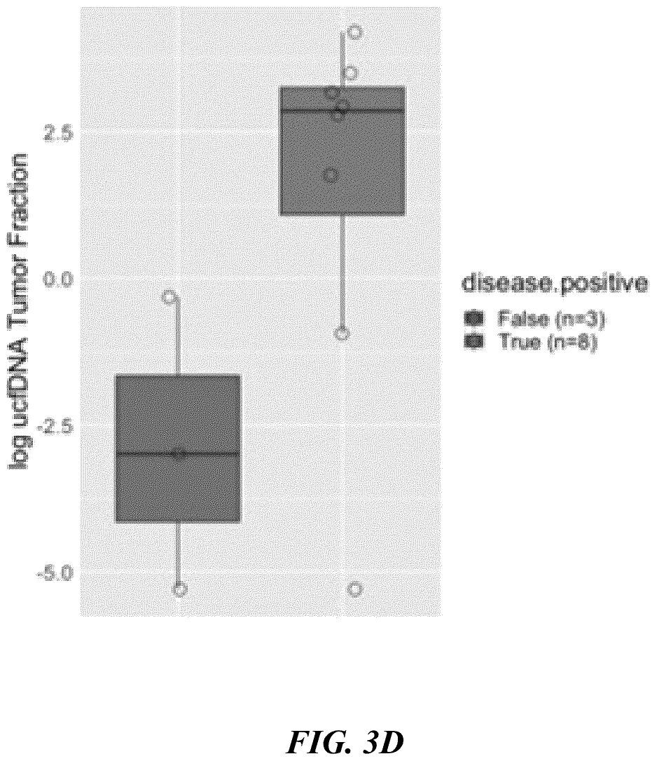

FIG. 2 shows and oncoplot of urinary tumor DNA genomic alterations in patients with NMIBC before repeat TURBT. Genes with non-synonymous (NS) mutations observed in index and rTURBT specimens identified in a minimum of two samples are reported. FIG. 3A shows WES+Variant frequencies between index and rTURBT sample. FIG. 3B shows variant overlap panel between index TURBT (blue), rTURBT (green), and urine samples (yellow). FIG. 3C shows utDNA positivity in patients with disease present at rTURBT. FIG. 3D shows tumor fraction of utDNA prior to rTURBT.

Eleven patients underwent rTURBT for high risk NMIBC. Residual tumor was detected at rTURBT in 8/11 (73%) patients. Fifty genes with NS mutations found in at least two samples were identified including TP53, PIK3CA, RBI, MYC, CDKN1A and ARID1A (FIG. 2, Table 3). A median of 146 (range 39-418) and 91 NS (range 2-312) mutations were identified in the index and re-TUR specimens, respectively. Concordance rates were 83% on average between primary and rTURBT, highest for patients upstaged to T2 disease, and lowest for patients harboring CIS. The tumor fraction of utDNA was higher in patients with residual disease (mean 2.5% vs. 0.2%, p=0.10) (FIG. 3D). Using tumor-fraction to predict MRD, the area under the receiver-operator curve AUC for this test was 0.85. Using an optimal threshold of >=3.3% tumor-fraction to define utDNA positivity, the test had a sensitivity of 75% at 100% specificity.

TABLE 3
List of genes with mutations detected by personalized MRD panel sequencing.

TP53, KMT2D, EP300, RB1, AFF3, CDKN1A, EPHB1, FAT1, FLNA, FRY, GAK, MSH2,
NEGR1, PCDHGA8, PIK3CA, SMARCA4, TSC1, AARS2, ABCA8, ABL1, ABL2, ACAP1,
ACPP, ACSS3, ACVR1, ACAMDEC1, AHNAK, BRCA2, CACNB1, DNAJB1, ERBB3,
FBXW7, FUBP3, KDM6A, MAP3K6, MEN1, NTRK1, PPP1R14C, PRKD1, TP53BP1

### CONCLUSION

Urinary tumor DNA shows promise as a surrogate for minimal residual disease and may predict TURBT pathology for NMIBC. Genomic alterations between index and rTURBT tumors are highly concordant in papillary tumors even in the setting of upstaging, which may aid in targeted intravesical or systemic therapy selection. Larger cohorts and long-term follow up is needed to determine if utDNA can risk-stratify patients.

### Example 2: Monitoring of a Subject for Minimal Residual Disease

A subject has a cancer and is subjected to a therapy to treat the cancer. A sample of the cancer (e.g., tumor cells, or from a biopsy, or from a resection of tumor tissue) is obtained from the subject. A mutational profile of the cancer is obtained via a whole exome assay. Specific mutations of the cancer are identified via the whole exome assay. Based on the cancer mutations in the profile, a custom panel of probes is made that is specific to the cancer.

After treatment, the subject is monitored for minimal residual disease. A biological sample (e.g., whole blood, plasma, urine, or urine cell pellet) is obtained from the subject. DNA from the sample is subjected to sequencing by using the custom panel (and optionally a panel containing previously identified hotspot mutations in cancers). Based on the DNA sequencing, the subject is identified as having a residual tumor. The subject is recommended to undergo additional therapeutic interventions.

### Example 3: Monitoring of a Subject after Transurethral Resection of Bladder Tumor

A subject has a bladder cancer and is subjected to transurethral resection of bladder tumor (TURBT) to remove the cancer. A mutational profile of the cancer is obtained via a whole exome assay. Specific mutations of the cancer are identified. Based on the cancer mutations in the profile a custom panel of probes is made.

The subject is observed to be cancer free and is monitored for minimal residual disease. A urine sample or urine cell pellet sample is obtained from the subject. Urinary cell free DNA is subjected to sequencing by using the custom panel and a panel containing previously identified hotspot mutations in cancers. Based on the urinary cell free DNA sequencing, the subject is identified as having a residual tumor. The subject is recommended to undergo additional therapeutic interventions including the use of repeat transurethral resection of bladder tumor (rTURBT).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-67. (canceled)

68. A method for identifying presence or an absence of minimal residual disease (MRD) in a subject, comprising:
(a) assaying deoxyribonucleic acid (DNA) molecules from a first biological sample obtained or derived from said subject at a first time point;
(b) detecting a set of biomarkers from said DNA molecules based at least in part on said assaying of (a), wherein said set of biomarkers comprise differentially expressed markers or variants;
(c) generating a plurality of probe nucleic acids that are customized for said subject, wherein said probe nucleic acids comprises sequences of at least a subset of said set of biomarkers;
(d) using said plurality of probe nucleic acids, sequencing cell free deoxynucleic acids (cfDNA) from a second biological sample obtained or derived from said subject at a second time point to detect the presence or absence of said subset of said set of biomarkers, wherein said sequencing is performed at a depth of at least 80×, wherein said second biological sample comprises a blood sample, a urine sample, or a urine cell pellet sample;
(e) computer processing said subset of said set of biomarkers to detect said presence or absence of minimal residual disease (MRD) in said subject.

69. The method of claim 68, wherein said first biological sample is selected from the group consisting of: a cell-free deoxyribonucleic acid (cfDNA) sample, a cell-free ribonucleic acid (cfRNA) sample, a plasma sample, a serum sample, a buffy coat sample, a peripheral blood mononuclear cell (PBMC) sample, a red blood cell sample, a urine sample, a urine cell pellet sample, a saliva sample, tissue biopsy, pleural fluid sample, peritoneal fluid sample, amniotic fluid sample, cerebrospinal fluid sample, lymphatic fluid sample, sweat sample, tear sample, semen sample, or any derivative thereof, and any combination thereof.

70. The method of claim 68, wherein said first biological sample or second biological sample comprises said plasma sample.

71. The method of claim 68, wherein the first biological sample or second biological sample comprise a urine cell pellet sample.

72. The method of claim 68, wherein said sequencing of (d) is performed at a depth of at least 10,000×.

73. The method of claim 68, wherein said sequencing of (e) comprises sequencing nucleic acids of a sample taken at said first time point, and sequencing nucleic acids of a sample taken at a second time point.

74. The method of claim 68, wherein said cancer is selected from the group consisting of: genitourinary cancer, prostate cancer, bladder cancer, and any combination thereof.

75. The method of claim 74, wherein cancer comprises a muscle invasive bladder cancer.

76. The method of claim 68, wherein said subject is asymptomatic for said cancer.

77. The method of claim 68, wherein the method comprises detecting said presence or absence of minimal residual disease in said subject at: (i) an accuracy of at least about 60%, (ii) a sensitivity of at least about 60%, (iii) a specificity of at least about 60%, (iv) a positive predictive value of at least about 60%, or (v) a negative predictive value of at least about 60%.

78. The method of claim 68, wherein said first biological sample is obtained or derived from said subject after performing a transurethral resection of bladder tumor.

79. The method of claim 68, further comprising identifying a clinical intervention for said subject based at least in part on said detected presence or said absence of said cancer,

80. The method of claim 79, wherein said clinical intervention is selected from the group consisting of: surgical resection, chemotherapy, radiotherapy, immunotherapy, adjuvant therapy, neoadjuvant therapy, androgen deprivation therapy, and a combination thereof.

81. The method of claim 68, wherein said set of biomarkers comprises one or more members selected from the group consisting of genes listed in Table 1, Table 2 or Table 3.

82. The method of claim 68, wherein (d) further comprises sequencing using a fixed plurality of probes wherein the probes of the fixed plurality of probes comprises probes that do not comprise sequences of said subset of said set of biomarkers.

83. The method of claim 82, wherein said fixed plurality of probes comprise one or more members selected from the group consisting of genes listed in Table 2.

84. The method of claim 68, wherein said set of biomarkers from said DNA molecules comprise tumor-associated alterations selected from the group consisting of: single nucleotide variants (SNVs), insertions or deletions (indels), and rearrangements.

85. The method of claim 68, further comprising determining, among said set of biomarkers, a mutant allele frequency of a set of somatic mutations.

86. The method of claim 85, further comprising determining a circulating tumor DNA (ctDNA) fraction, or an abnormality score based at least in part on said mutant allele frequency of a set of somatic mutations.

87. The method of claim 68, further comprising determining a tumor mutational burden (TMB) of said cancer of said subject.
